(19) United States
(12) Patent Application Publication
(10) Pub. No.: US 2008/0275135 A1
(43) Pub. Date: Nov. 6, 2008

(54) Resorcinol Derivatives and Their Use for Lowering Blood Pressure

(75) Inventors: Raphael Mechoulam, Jerusalem (IL); Yehoshua Maor, Brighton, MA (US); Lumir Hanus, Jerusalem (IL); Michal Horowitz, Jerusalem (IL)

Correspondence Address: Fleit Gibbons Gutman Bongini & Bianco PL, 21355 EAST DIXIE HIGHWAY, SUITE 115, MIAMI, FL, 33180, US

(73) Assignee: YISSUM RESEARCH DEVELOPMENT COMPANY OF THE HEBREW, Jerusalem (IL)

(21) Appl. No.: 11/570,958

(22) PCT Filed: Jun. 20, 2005
(86) PCT No.: PCT/IL05/00659

§ 371 Date: May 7, 2007

(30) Foreign Application Priority Data
Jun. 20, 2004 (IL) 162636

(51) Int. Cl. C07C 39/10; A61K 31/05; A61P 9/12

(52) U.S. Cl. 514/731; 568/763

(57) ABSTRACT

Disclosed are synthetic cannabinoids, namely resorcinol derivatives, which reduce blood pressure without having psychotropic effects. Pharmaceutical compositions for the treatment of high blood pressure related conditions, as well as methods of treating the same utilizing as active agent the compounds of the invention are also disclosed herein. Preferred active compounds are those which bear a long side chain on the C₅ position and a terpenoid chain on the C₂ position.

### FIELD OF THE INVENTION

The present invention refers to the field of drug development and heart disease. More specifically, the present invention describes novel compounds, mainly resorcinol derivatives, and their uses in the regulation of blood pressure.

### BACKGROUND OF THE INVENTION

All publications mentioned throughout this application are fully incorporated herein by reference, including all references cited therein.

High blood pressure, or the condition known as hypertension is characterized by a blood pressure that remains persistently higher than it should normally be. It occurs when pressure builds up in the arteries as the heart pumps the blood round. It is a very problematic condition since usually there are no symptoms and, therefore, it has been named as a silent killer.

Arterial Hypertension is characterized by systolic blood pressure ≧140 mmHg and/or diastolic ≧90 mmHg. Arterial hypertension is often (if not always) one of the causes of congestive heart failure (CHF), a condition characterized by several symptoms amongst which are fatigue, shortness of breath, swelling of legs and ankles, rapid heartbeat, and others.

Another condition involving high blood pressure is Isolated Hypertension, or Isolated Systolic Hypertension (also known as ISH), wherein systolic blood Pressure ≧140 mmHg with diastolic blood pressure <90-95 mmHg. Although in the past it was thought that the low diastolic pressure might be a favorable feature, nowadays it is known that this is not the case. ISH is usually an indication of diseased vessels, which implicates bad prognosis.

Hypertension is also associated with diabetes, or with conditions that result in renal failure.

Current therapy for hypertension (or high blood pressure) is based on diuretics, β-blockers, long-acting Ca₂₊ blockers, ACE inhibitors, angiotensin II receptor blockers and α-adrenergic blockers. However, not all patients are responsive to the available drugs and therefore, new types of drugs are needed.

Resorcinols are 1,3 dihydroxybenzene derived organic compounds. All plant cannabinoids are resorcinol-derived compounds in which the C-5 side chain is an alkyl chain of 3 to 5 carbons and the C-2 substitution is a monoterpene (a ten carbon compound derived from many possible known natural terpenes). The present inventors have generated resorcinol-derived compounds, like for example 2-geranyl-5-(1,1-dimethylheptyl)-resorcinol (also known as cannabigerol-dimethylheptyl), and evaluated its biological activity.

Both endogenous and various synthetic cannabinoids are known to have cardiovascular side effects, in particular the ability to induce bradycardia and hypotension, i.e., to reduce blood pressure (Hogestatt, E. D. and Zygmunt, P. M. (2002) Prostaglandins, Leukotrienes and Essential Fatty Acids 66(2&3), 343-351]. However they all possess central psychotropic effects and are therefore not suitable as drugs for the treatment of cardiovascular conditions. Attempts to separate the hypotensive action of Δ9-THC from its psychotropic properties have achieved only partial success [Zaugg and Kyncl (1983) J Med. Chem. 26(2): 214-7]. A new pharmacological target (Abn-CBD sensitive receptor) was tentatively shown to be present in the endothelium of peripheral blood vessels [Jarai et al. (1999) Proc. Natl. Acad. Sci. USA 96(24): 14136-14141]. This putative receptor induces hypotension when activated by Abn-CBD, (−)-4-(3-3,4-trans-p-menthadien-1,8)-yl-olivetol, a compound which results from the transposition of the phenolic hydroxyl group and the pentyl side chain of cannabidiol.

In the present study, the inventors have synthesized new resorcinol derivatives, and found that these compounds have anti-hypertensive properties without having psychotropic activity.

Thus, it is an object of the present invention to provide compounds, specifically novel resorcinol derivatives, and their use as drugs for the regulation of blood pressure. Other uses and objects of the invention will become apparent as the description proceeds.

### SUMMARY OF THE INVENTION

The present inventors generated novel resorcinol derivatives, and studied their function in the cardiovascular system. Unexpectedly, the present inventors found that resorcinol derivatives which possess a long side chain (preferably dimethylheptyl) on the C₅ position and a terpenoid side chain (preferably geranyl) on the C₂ position are able to reduce blood pressure in an experimental model system, without having psychotropic effects.

Thus, in a first aspect, the present invention provides a composition comprising as active ingredient a compound of general formula (I):

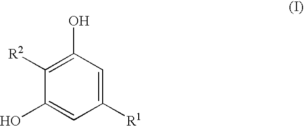

wherein
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and R⁴ is a straight or branched alkyl chain of 1 to 5 carbon atoms; and
R² is a non-cyclic terpenoid comprising from 10 to 30 carbon atoms; and further comprising at least one pharmaceutically acceptable additive, diluent or carrier.

In one embodiment of the composition of the invention, R¹ is a straight alkyl chain of 5 to 8 carbon atoms, optionally substituted with one methyl group.

In a second embodiment, R² is selected from the group consisting of geranyl, optionally substituted with one —OH, and farnesyl optionally substituted with one —OH.

In a most preferred embodiment of the composition of the invention, R¹ in formula (I) is dimethylheptyl and R² is geranyl.

The composition of the invention is intended for medical use.

Specifically, in one further embodiment of the composition of the invention, said composition is intended for the treatment of a disorder selected from high blood pressure, and conditions associated therewith. Generally, the composition of the invention is intended for the treatment of a disorder selected from the group consisting of hypertension, isolated hypertension, congestive heart failure, and left ventricular hypertrophy.

The composition of the invention is particularly intended for lowering systolic blood pressure.

In a second aspect, the present invention provides a compound of formula (I):

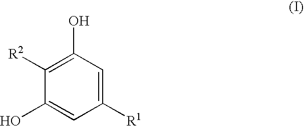

wherein:
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms; with the proviso that when R¹ is isononyl, R² is not geranyl.

In one embodiment of the compound of the invention, R¹ is a straight alkyl chain of 5 to 8 carbon atoms, optionally substituted with one methyl group.

In a second embodiment, R² is selected from geranyl optionally substituted with one —OH, and farnesyl optionally substituted with one —OH.

In a further aspect the present invention provides a pharmaceutical composition comprising as active ingredient a compound as defined above, more specifically, a compound of formula (I):

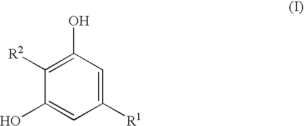

wherein:
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms; with the proviso that when R¹ is isononyl, R² is not geranyl.

In one embodiment, said pharmaceutical composition is for medical use. Particularly, said composition is for the prevention and/or treatment of any one of high blood pressure and conditions resulting therefrom and/or associated therewith, and specifically for lowering systolic blood pressure.

The pharmaceutical composition of the invention is also intended for vasodilation of blood vessels.

In yet a further aspect, the present invention provides the use of a compound of general formula (I):

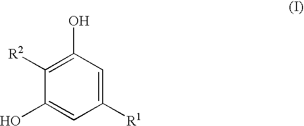

wherein
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms;
in the preparation of a composition for lowering systolic blood pressure and/or for the prevention and/or treatment of any one of high blood pressure and conditions resulting therefrom and/or associated therewith.

In one embodiment of the use of the compound of the invention, R¹ is dimethylheptyl and R² is geranyl.

Lastly, the invention provides a method of prevention and/or treatment of conditions resulting from high blood pressure, comprising administering a therapeutically effective amount of a compound of general formula (I):

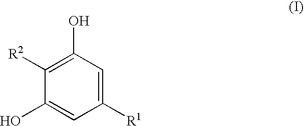

wherein
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms; and isomers or compositions thereof; to a subject in need.

In one specific embodiment of the compound to be administered in the method of the invention, R¹ is dimethylheptyl and R² is geranyl.

In another embodiment of the method of the invention, said conditions are selected from one of hypertension, isolated hypertension, congestive heart failure, left ventricular hypertrophy, and disorders alike. Treatment of high blood pressure is also sought for in patients suffering from renal failure.

### BRIEF DESCRIPTION OF THE FIGURES

FIG. 1: Effect of cannabigerol, dimethylheptyl (also referred to as Compound 1) on systolic blood pressure.

The graph shows the effect of different concentrations (2.5, 5, 7.5 and 10 mg/kg) of CBG-DMH (cannabigerol dimethylheptyl, Compound 1) on blood pressure (BP) in mmHg units. Reduction in blood pressure is already observed at the lowest concentration (2.5 mg/kg), and the effect becomes much more pronounced at 5 mg/kg of CBG-DMH.

Abbreviation: Init. BP; initial blood pressure.

FIG. 2A-2B: Effect of Compound 1 on aortic ring relaxation.

FIG. 2A: Endothelium dependency of CBG-DMH-induced vasorelaxation in rat aortic rings.

FIG. 2B: O-1918 antagonist does not antagonize the relaxation-potential of CBG-DMH.

Abbreviations: init. tens., initial tension; int. art., intact artery; den. art., denuded artery.

### DETAILED DESCRIPTION OF THE INVENTION

The present inventors have generated novel compounds of resorcinol derivatives, having general formula (I):

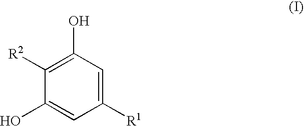

wherein
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms.

Preferably, R¹ and R² are as follows:
R¹ is a straight alkyl chain of 5 to 8 carbon atoms, optionally substituted with one methyl group;
R² is selected from geranyl optionally substituted with one —OH, and farnesyl optionally substituted with one —OH.

By compounds of formula I it is also meant the geometric isomers around double bonds (where applicable) and enantiomers in compounds that contain an asymmetric center.

In studying the biological activity of these compounds, the inventors found that they were effective in lowering blood pressure in an experimental model system. This effect was particularly evidenced in compounds which possess a long side chain (preferably dimethylheptyl) on the C₅ position and a terpenoid side chain (preferably geranyl) on the C₂ position.

Thus, the present invention provides a pharmaceutical composition comprising as active agent a compound of general formula (I):

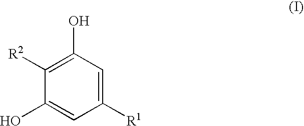

wherein
R¹ is selected from:
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms; and isomers thereof; further comprising at least one pharmaceutically acceptable additive, vehicle, diluent or carrier.

In a preferred embodiment, the composition of the invention further comprises a vehicle which is a mixture of ethanol: Emulphor®:PBS (at 1:1:18 v/v ratio).

Poly(ethylene glycol) and cyclodextrins of various types, like alkylated beta-cyclodextrin, for example, are also suitable carriers for the composition of the invention.

Other possible diluents are an aqueous cosolvent solution, comprising a pharmaceutically acceptable cosolvent, a micellar solution prepared with natural or synthetic ionic or nonionic surfactants, or a combination of such cosolvent and micellar solutions, etc.

Carriers may consist essentially of a solution of ethanol, a surfactant or water, or an emulsion comprising triglycerides, lecithin, glycerol, emulsifiers, antioxidants, water, etc.

The composition of the invention may further comprise an excipient selected among a Carrier, a disintegrant, a lubricant, a stabilizer, a flavoring agent, another pharmaceutical effective compound, etc.

The composition of the invention may be used in combination with anti-fibrinolytic agents.

The preparation of pharmaceutical compositions is well known in the art and has been described in many articles and textbooks, see e.g., Remington's Pharmaceutical Sciences, Gennaro A. R. ed, Mack Publishing Co., Easton, Pa., 1990, and especially pp. 1521-1712 therein.

It should be noted that the compound cannabigerol dimethylheptyl (in which R¹ is a dimethylheptyl and R² is geranyl, also referred to herein below as Compound 1), has been previously described [Baek, S. et al. (1995) Bull. Korean Chem. Soc. 16, 281-284].

Thus, the present invention provides compounds of general formula (I):

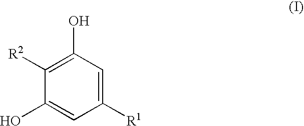

wherein:
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms;
with the proviso that when R¹ is isononyl, R² is not geranyl.

Compositions comprising the above-defined compound are also provided in the present invention, which may be intended for medical use.

Therefore, the invention provides a method of prevention and/or treatment of conditions resulting from high blood pressure, comprising administering a therapeutically effective amount of a compound of the general formula (I):

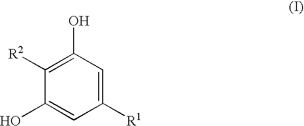

wherein:
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and R⁴ is a straight alkyl chain of 1 to 5 carbon atoms; and
R² is a non-cyclic terpenoid comprising from 10 to 30 carbon atoms; and isomers or compositions thereof; to a subject in need.

In one specific embodiment of the compound to be administered in the method of the invention, R¹ is dimethylheptyl and R² is geranyl.

Said therapeutic effective amount, or dosing, is dependent on severity and responsiveness of the condition to be treated, and can be determined by standard clinical techniques, with the course of treatment lasting from several days to several months, or until a cure is effected or a diminution of the condition is achieved. In general, the medical personnel in charge of the subject in need of the treatment can easily determine optimum dosages, dosing methodologies and repetition rates.

In addition, in vitro assays as well in vivo experiments may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems. The amount must be sufficient to lower systolic blood pressure to levels considered normal for the specific subject under treatment. In general, optimal dosages vary between 1 to 10 mg/kg, and may reach up to between 80 and 300 mg/dose, preferably 100 mg/dose.

Various methods of administration may be used for delivering the compounds of the invention or a composition thereof to a subject in need. The compounds of the invention, or compositions thereof, may be delivered via intravenous (i.v.), intramuscular (i.m.) intraperitoneal (i.p.) injections, orally (in liquid form or prepared as dosage unit forms like capsules, tablets, granules, pills, lozenges, etc.). For administration by inhalation, the compositions are conveniently delivered in the form of drops or aerosol sprays.

The compositions of the invention may be used therapeutically alone, or in combination with other drugs.

The mechanism of blood pressure reduction by the compounds of the invention, and particularly Compound I, is through a novel pathway different from the mechanisms of the hypertension drugs used in patients so far. Hence it can be used in combination with other drugs as the present compounds and the anti-hypertensive drugs currently available in the market are likely to complement each other. This novel mechanism involves a new cannabinoid receptor which does not lead to psychoactivity and is activated by the endogenous arachidonoyl serine as previously described by the inventors [Milman, G. et al. (2004) Arachidonoyl-serine, an endocannabinoid-like bioactive constituent of rat brain. Abstract presented at the 2004 Symposium on the Cannabinoids, organized by the International Cannabinoid Research Society, in Paestum, Italy].

The findings presented herein are extremely useful for the development of novel anti-high blood pressure drugs. Most importantly, the compounds of the present invention do not bind to cannabinoids receptors (as described in Example 3) and do not demonstrate any measurable psychotropic effects (as described in Example 4). This is of major significance for testing said new compounds in human subjects, who suffer from the above-cited conditions and are likely to benefit from the properties of these compounds.

Thus, the present invention provides a method and compositions for lowering systolic blood pressure, comprising administering a therapeutically effective amount of a compound of the general formula (I) as described above.

Therefore, the present invention provides compounds which are to be used in the treatment or in the preparation of pharmaceutical compositions for the treatment of hypertension, isolated hypertension, pulmonary hypertension, congestive heart failure, left ventricular hypertrophy, atherosclerosis, stroke, peripheral vascular disease, conditions of reduced blood vessel patency, and disorders alike. Treatment of high blood pressure is also sought for in patients suffering from renal failure.

The present invention is defined by the claims, the contents of which are to be read as included within the disclosure of the specification.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Example 1

### Synthesis of Resorcinol Derivatives

Resorcinol derivatives were synthesized essentially, as previously described [Baek S. et al. (1995) Bull. Koreain Chem. Soc. 16, 281-284]. These compounds were prepared by the condensation of a resorcinol derivative, substituted with an R¹ side chain at C₅, with an allylic alcohol R²—OH, preferably a terpenoid allylic alcohol) in the presence of BF₃ etherate to yield the desired product. The general formula of the resorcinol derivatives produced can be represented by formula (I) below:

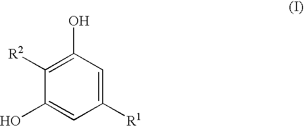

Wherein R¹ stands for either one of:
a. a straight or branched alkyl of 7 to 12 carbon atoms;
b. a group —O—R^{a}, where R^{a} is a straight or branched alkyl of 5 to 9 carbon atoms, or a straight or branched alkyl substituted at the terminal carbon atom by a phenyl group; or
c. a group —CH₂)ₙ—O-alkyl, where n is an integer from 1 to 7 and the alkyl group contains 1 to 5 carbon atoms.
   R² stands for a non-cyclic terpenoid carbon chain such as geranyl, farnesyl, and related non-cyclic terpenes and their isomers as well as other non cyclic paraffinic or olefinic carbon chains.

Essentially, the compounds were synthesized as follows:

1. To a stirred suspension of silica gel (20 g) in dry CH₂Cl₂ (100 ml) under N₂, BF₃ etherate (2 ml) was added.
2. The mixture was stirred for 15 minutes at room temperature, and a solution of 1′, 1′-dimethyl heptyl resorcinol (2.36 g) and geraniol (2.3 g), in 20 ml of dry CH₂Cl₂, was added in one portion.
3. The reaction mixture was stirred at room temperature for 48 hours.
4. A saturated solution of NaHCO₃ was added (200 ml) to the mixture.
5. The mixture was then separated and the aqueous layer was extracted three times with dichloromethane.
6. The combined organic extracts were dried and evaporated. After the evaporation the final weight was 5.37 grams.
7. The compound was purified by column chromatography on silica gel to give 4.3 g pure cannabigerol dimethylheptyl (denominated Compound 1, wherein R¹=1,1-dimethylheptyl; R²=geranyl).

¹H NMR: (CDCl₃), δ 0.85 (3H, t, CH₃), 1.20 (6H, s, two CH₃), 1.59, 1.67, 1.80 (9H, s, olefinic CH₃), 3.42 (2H, d, J=8.0 Hz, C-8H), 5.04-4.94 (2H, m, olefinic H), 6.37 (2H, s, arom H).

### Example 2

### Resorcinol Derivatives Induce Blood Pressure Reduction

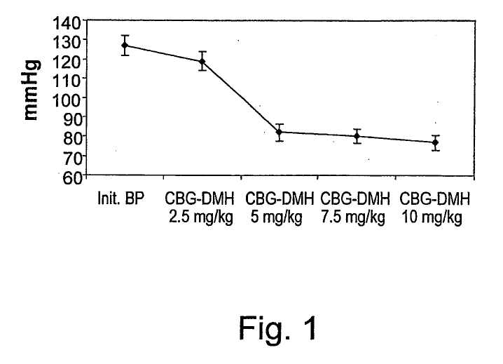

The protocol for testing blood pressure was applied essentially as previously described [Shochina, M. and Horowitz, M. (1989) J. Therm. Biol. 14, 109 113]. Adult male Sabra rats weighing between 225 to 250 g had their femoral vein cannulated for intravenous (i.v.) drug administration. Anesthesia was induced by the intraperitoneal (i.p.) injection of pentobarbital sodium 6%, 60 mg/kg. The femoral artery was cannulated and a catheter (PE 10 cannulae) was connected to a pressure transducer for continuous monitoring of blood pressure with a physiograph (AcKnowledge program). After a 30 minute stabilization period, the animals were injected with either vehicle (sahine:ethanol:Emulphor® 18:1:1) or the drug (Compound 1, as described above, varying from 1 to 10 mg/kg) injected in bolus i.v. in volumes ≧500 μl. The changes in blood pressure were monitored for 60 minutes. The data in FIG. 1 indicates the mean systolic blood pressure observed over this period, and shows a clear reduction of the systolic blood pressure of the animal tested. Compound 1 caused hypotension in rats in doses of 5 mg/kg without causing change in the heart rate. The effect was antagonized by CBD in similar doses. The hypotensive activity of Compound 1 leads the way to a new class of atypical cannabinoids with no psychotropic activity and with a mechanism of action differing from the anti-hypertensive drugs known to date.

### Example 3

### Tests for Compound 1 Binding to Cannabinoid Receptors

The protocol for cannabinoid receptor binding has been previously described [Devane W. A. et al. (1992) Science 258, 1946-1949]. The high affinity receptor probe [³H]HU-243, with a dissociation constant of 45+7 pM, was incubated with synaptosomal membranes (3 to 4 μg) for CB1 assays and/or transfected cells for CB2 assays, for 90 minutes at 30° C. with the different concentrations of resorcinol derivatives, specifically Compound 1, or with the vehicle alone (fatty-acid-free bovine serum albumin at a final concentration of 0.5 mg/ml). Bound and free radioligand were separated by centrifugation. The data were normalized to 100% of specific binding, which was determined with 50 nM unlabeled HU-243. The Ki value was determined using the program GraphPad Prism (Version 3.02) which follows the Cheng-Prusoff equation. A sigmoid dose-response (variable slope) built-in equation in this Prism program was used to fit the curves. The results obtained showed no binding between Compound 1 and either cannabinoid receptor CB1 or CB2 (data not shown).

### Example 4

### Test for Psychotropic Activity of the Resorcinol Derivatives

The protocol for cannabinoid receptor binding has been previously described [Fride, E. and Mechoulam, R. (1993) Eur. J. Pharmacol. 231, 313-314]. A standard test for cannabinoid psychotropic activity is the Tetrad Test. This assay consists of four separate tests: (i) Ring immobility (catalepsy) test, which measures the percent of time over 4 minutes that mice remain motionless on a ring (5.5 cm diameter); (ii) Open field test, which measures horizontal (locomotor) and vertical (rearing) activity; (iii) Hypothermia (AOC); and (iv) Antinociception (hot plate latency). For all these tests, the resorcinol derivatives described in the invention were negative, i.e., they did not present any psychotropic activity (data not shown).

### Example 5

### Compound 1 has Vaso-Relaxant Properties

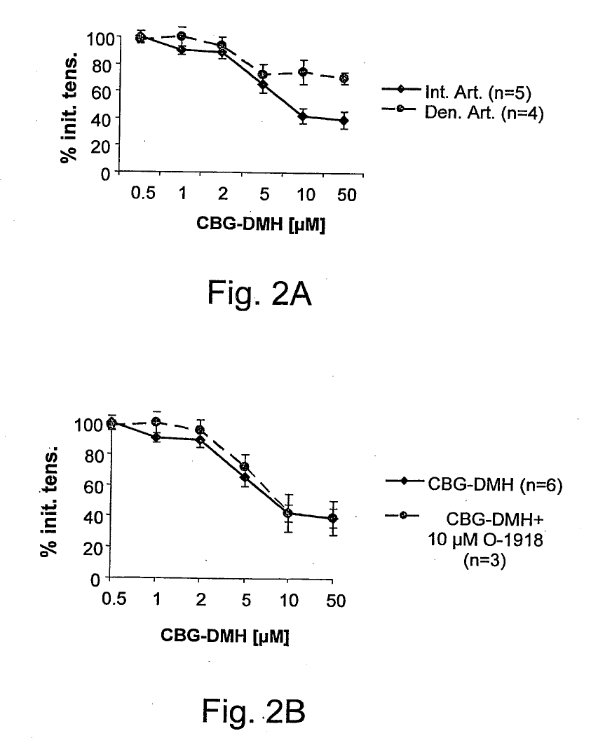

Abdominal aortic rings of male Sabra rats of 300 g average body weight were obtained from the animal facility of the Hebrew University of Jerusalem, campus Ein Kerem, Jerusalem, Israel. The animals were anaesthetized by an intra-peritoneal injection of pentobarbital (50 mg kg⁻¹). After thoracotomy, the aorta was excised, transferred to a dish filled with Krebs-Henseleit buffer (composition in mM: NaCl 150.0, KCl 5.4, MgSO₄ 1.17, NaH₂ PO₄ 1.18, NaHCO₃ 6.0, CaCl₂ 1.0, HEPES 20.0, glucose 5.5, pH 7.4), cleared of periadventitial tissue, and cut into ring segments (3 mm in length). Only segments of the abdominal aorta were used. Two stainless-steel hooks were carefully passed through the lumen of each ring. One hook was connected to an isometric force transducer (BIOPAC Instruments, Goleta, Calif.) to measure tension in the vessels. The rings were placed in a 10-ml organ bath, gassed with 5% CO₂ in O₂ and maintained at 37° C. The rings were stretched until an optimal basal tension of 1.0 g. After this tension was achieved they were allowed to equilibrate for 60 minutes with the bath fluid being changed every 15-20 min. Baseline tension usually stabilized within 60 min of mounting, during which time there were four replacements of bathing solution. The segment was then pre-contracted by 5 μM phenylephrine. Addition of CBG-DMH in cumulative doses led to relaxation of the contracted segments (FIG. 2A). The levels of relaxation were then measured. The endothelial function was assessed by testing the relaxant effect of acetylcholine (10 nM-40.1 mM) on aortic rings pre-contracted with phenylephrine. Failure of acetylcholine to elicit relaxation of aortic rings previously subjected to rubbing of the intimal surface was taken as evidence of endothelium removal. Concentration-response curves were generated by cumulative addition of the agonist. Whenever antagonists (PTX 0.5 μg/ml, SR-141716 A, SR-144528 or 10 μM of 0-1918) were used, they were added 20 min before the agonist (FIG. 2B). All experiments were conducted with aluminum foil-covered organ bath to prevent light-induced degradation of the drugs.

FIG. 2A shows that endothelium dependency of CBG-DMH-induced vasorelaxation could be detected in aortic rings with a maximum vasorelaxation of 55% (+4%) in the intact artery as opposed to 25% (+1%) in the denuded artery. The aortic vasorelaxant effect was inhibited in the presence of pertussis toxin (0.5 μg/ml, data not shown). Preliminary data suggests that CBG-DMH also functions as a vasodilator.

Interestingly, treatment of the aortic rings with the antagonist 0-1918, followed by CBG-DMH did not result in antagonism (FIG. 2B), suggesting that the mechanism of action of CBG-DMH may differ from Abn-CBD, which has been shown to be antagonized by 0-1918 [Offertaler, L. et al. (2003) Mol. Pharmacol. 63(3): 699-705].

1. A pharmaceutical composition comprising as active ingredient a compound of formula (I): 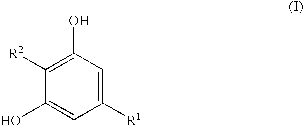
wherein
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and R⁴ is a straight or branched alkyl chain of 1 to 5 carbon atoms; and
R² is a non-cyclic terpenoid comprising from 10 to 30 carbon atoms;
and further comprising at least one pharmaceutically acceptable additive, diluent or carrier.

2. The composition according to claim 1, wherein R¹ is a straight alkyl chain of 5 to 8 carbon atoms, optionally substituted with one methyl group.

3. The composition according to claim 1, wherein R² is selected from geranyl optionally substituted with one —OH, and farnesyl optionally substituted with one —OH.

4. The composition according to claim 1, wherein R¹ in formula (I) is dimethylheptyl and R² is geranyl.

5. (canceled)

6. (canceled)

7. (canceled)

8. (canceled)

9. A compound of formula (I): 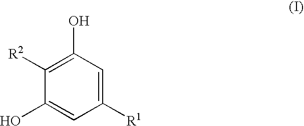
wherein:
R¹ is selected from
a. straight or branched alkyl chain of 7 to 12 carbon atoms;
b. —O—R³, where R³ is a straight or branched alkyl chain of 5 to 9 carbon atoms, optionally substituted by one phenyl group; and
c. —(CH₂)ₙ—O—R⁴, where n is an integer from 1 to 7, and 144 is a straight alkyl chain of 1 to 5 carbon atoms; and
R² is a non-cyclic terpenoid comprising from 10 to 30 carbon atoms;
with the proviso that when R¹ is isononyl, R² is not geranyl.

10. A compound according to claim 8, wherein R¹ is a straight alkyl chain of 5 to 8 carbon atoms, optionally substituted with one methyl group.

11. A compound according to claim 8, wherein R² is selected from geranyl optionally substituted with one —OH, and farnesyl optionally substituted with one —OH.

12. A pharmaceutical composition comprising as active ingredient a compound according to claim 9, and further optionally comprising at least one pharmaceutically acceptable additive, diluent or carrier.

13. A method for the treatment of a disorder selected from high blood pressure, and conditions associated therewith, said method comprising administering a therapeutically effective amount of the pharmaceutical composition of claim 1 to a subject in need.

14. A method for the treatment of a disorder selected from the group consisting of hypertension, isolated hypertension, congestive heart failure, and left ventricular hypertrophy, said method comprising administering a therapeutically effective amount of the pharmaceutical composition of claim 1 to a subject in need.

15. A method for lowering systolic blood pressure, said method comprising administering a therapeutically effective amount of the pharmaceutical composition of claim 1 to a subject in need of lowering systolic blood pressure.

16. A method for vasodilation of blood vessels, said method comprising administering a therapeutically effective amount of the pharmaceutical composition of claim 1 to a subject in need of vasodilation of blood vessels.
